(19) United States
(12) Patent Application Publication
(10) Pub. No.: US 2006/0206137 A1
(43) Pub. Date: Sep. 14, 2006

(54) Balloon structure and balloon catheter

(75) Inventor: Gianluca Gazza, Avenue Princesse Grace (MC)

Correspondence Address: SHOEMAKER AND MATTARE, LTD, 10 POST OFFICE ROAD - SUITE 110, SILVER SPRING, MD, 20910, US

(73) Assignee: INVATEC S.r.1., Roncadelle (Brescia) (IT)

(21) Appl. No.: 10/553,973

(22) PCT Filed: Apr. 24, 2003
(86) PCT No.: PCT/IB03/01531

§ 371 Date: Oct. 19, 2005

(51) Int. Cl. A61M 29/00

(52) U.S. Cl. 606/194

(57) ABSTRACT

An inflatable balloon structure (10) for catheters, such as a catheter for angioplasty or for depositing an endolumenal prosthesis or stent in a duct, for example, a vascular duct, the balloon structure being of predominant longitudinal extent with a proximal end and a distal end, and being suitable for performing an expansion in an object to be dilated, the balloon comprising a wall which has, transverse the longitudinal extent, at any point, an annular cross-section delimited externally by an outer surface which, at least in an intermediate portion thereof, is suitable for coming into contact with the object to be dilated, and internally by an inner surface which delimits an inflation chamber, in which at least one wall cavity (19) is provided in the wall and is formed within the annular cross-section which delimits the inflation chamber so as to be disposed between the outer surface and the inner surface, the cavity extending without interruptions and/or openings, longitudinally relative to the balloon structure, between the proximal end and the distal end.

The subject of the present invention is a balloon structure and a catheter comprising the balloon structure.

The present invention relates in particular to an inflatable balloon structure for catheters such as, for example, a catheter for angioplasty or for depositing an endolumenal prosthesis or stent in a duct, for example, a vascular duct.

One of the main requirements in the field is to provide balloon structures which, when inserted in a catheter, allow paths to be followed by the catheter-even if the paths are tortuous as those of blood vessels, in particular but not exclusively coronary vessels, may be.

A further requirement of these structures is for the catheter provided with the structure to be capable of being oriented correctly, even when there are branches in the vascular duct, in particular but not exclusively enabling the catheter to perform even quite abrupt changes in direction.

Some solutions proposed for satisfying these requirements are known but they are not completely satisfactory.

For example, catheters with balloon structures which at least partially satisfy these requirements are known from U.S. Pat. No. 5,061,273 (YOCK), U.S. Pat. No. 4,762,129 (BONZEL), U.S. Pat. No. 4,545,390 (LEARY), U.S. Pat. No. 4,299,226 (BANKA), U.S. Pat. No. 4,195,637 (GRUNTZIG), U.S. Pat. No. 4,877,031 (CONWAY), U.S. Pat. No. 4,921,483 (WIJAY), U.S. Pat. No. 4,944,745 (SOGARD), U.S. Pat. No. 4,964,853 (SUGIYAMA), U.S. Pat. No. 5,032,113 (BURNS), U.S. Pat. No. 5,090,958 (SAHOTA), U.S. Pat. No. 5,330,499 (KANESAKA), U.S. Pat. No. 6,530,938 (LEE), U.S. Pat. No. 6,491,619 (TRAUTHEN), U.S. Pat. No. 5,895,405 (INDERBITZEN), U.S. Pat. No. 6,027,475 (SIRHAN), U.S. Pat. No. 5,980,486 (ENGER), U.S. Pat. No. 5,743,875 (SIRHAN),. U.S. Pat. No. 5,496,346 (HORZEWSKI), U.S. Pat. No. 5,472,425 (TEIRSTEIN).

A further important requirement in the field is to provide a balloon structure which permits the production of catheters with large balloon-inflation cavities or, in other words, with inflation chambers that are free of large obstructions and which permit rapid inflation and, above all, rapid deflation of the balloon so as to re-establish free circulation quickly when the catheter is used to dilate the walls of a blood vessel or to deposit an endolumenal prosthesis therein.

Some solutions proposed for satisfying these requirements, which conflict with the former above-mentioned requirements, are known but are not entirely satisfactory.

For example, catheters with balloon structures which at least partially satisfy these requirements are known from U.S. Pat. No. 4,983,167 (SAHOTA), U.S. Pat. No. 5,315,747 (SOLAR), U.S. Pat. No. 6,394,995 (SOLAR), U.S. Pat. No. 6,007,517 (AMDERSON), U.S. Pat. No. 5,413,557 (SOLAR), U.S. Pat. No. 5,383,853 (JUNG), U.S. Pat. No. 5,690,642 (OSBORNE), U.S. Pat. No. 5,921,958 (RESSEMANN), U.S. Pat. No. 5,458,639 (TSUKASHIMA), U.S. Pat. No. 5,980,484 (RESSEMANN), U.S. Pat. No. 5,575,771 (WALINSKY), U.S. Pat. No. 5,549,557 (STEINKE), U.S. Pat. No. 5,549,556 (NDONDO-LAY), U.S. Pat. No. 5,569,199 (SOLAR), U.S. Pat. No. 5,520,647 (SOLAR), U.S. Pat. No. 5,882,336 (JANACEK).

However, none of the solutions cited above succeeds in satisfying all of the requirements simultaneously.

The object of the present invention is to devise and make available a balloon structure and a balloon catheter which satisfy the above-mentioned requirements.

This object is achieved by means of a balloon structure formed in accordance with claim 1.

This object is also achieved by a balloon catheter according to claim 34.

Further embodiments of the balloon structure and of the catheter according to the invention are described in the dependent claims.

Further characteristics and the advantages of the balloon structure and of the catheter according to the invention will become clear from the following description of preferred embodiments which are given by way of non-limiting example with reference to the appended drawings, in which:

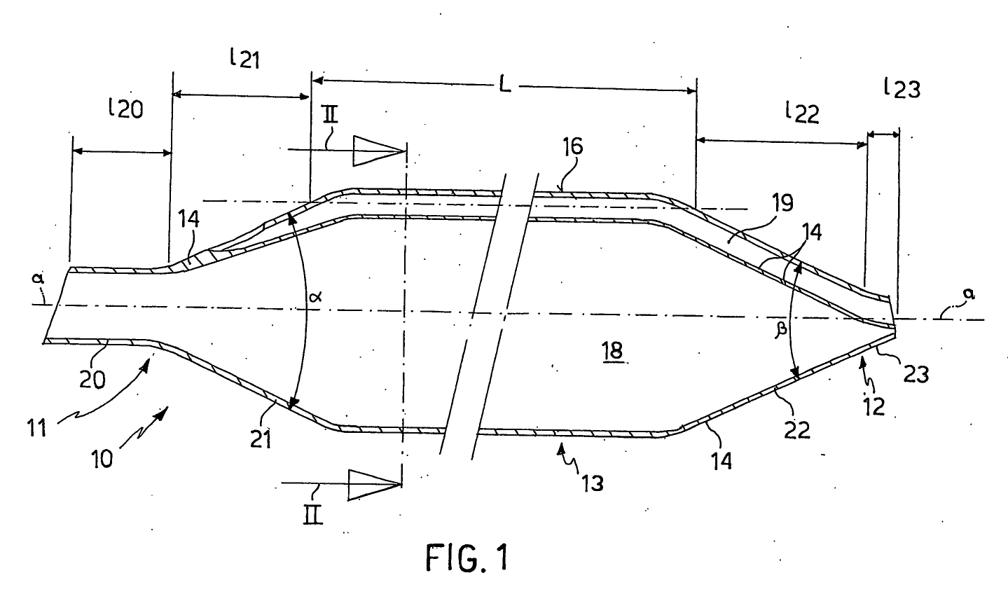

FIG. 1 is a section through an embodiment of a balloon structure,

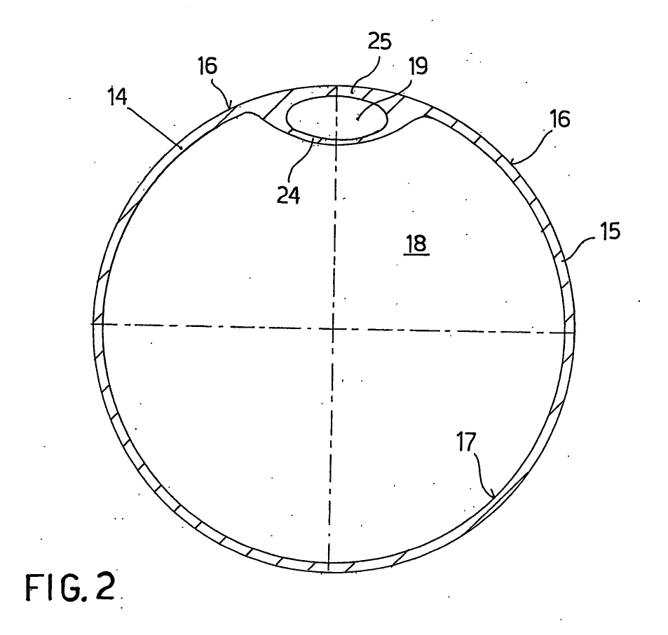

FIG. 2 is a section transverse the axis of the balloon structure of FIG. 1, taken on the line II-II,

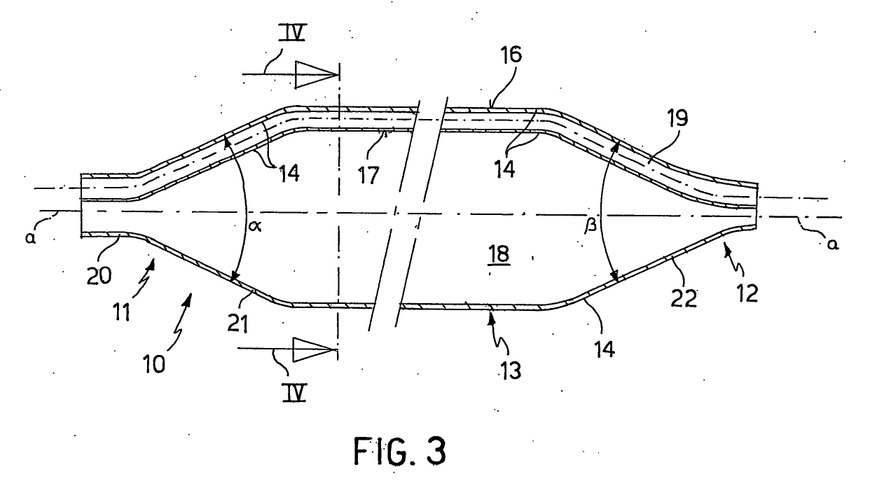

FIG. 3 is a section through a second embodiment of the balloon structure,

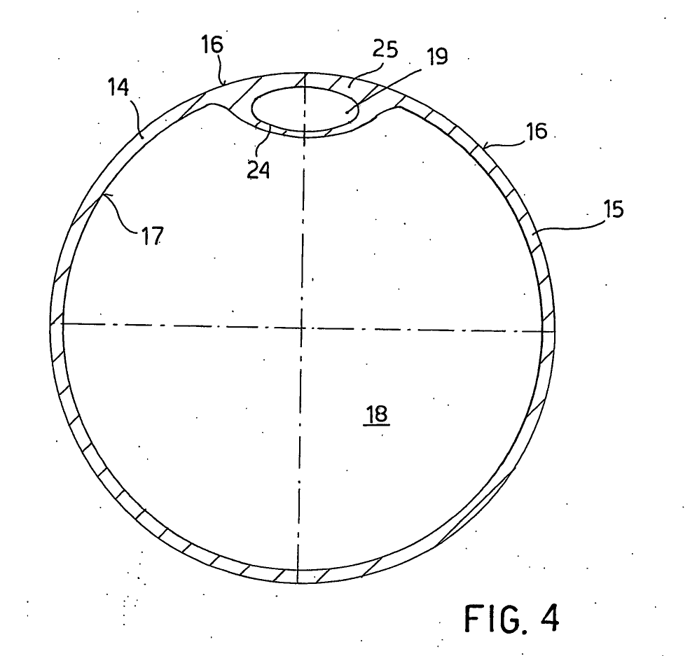

FIG. 4 is a section transverse the axis of the balloon structure of FIG. 3, taken on the line IV-IV,

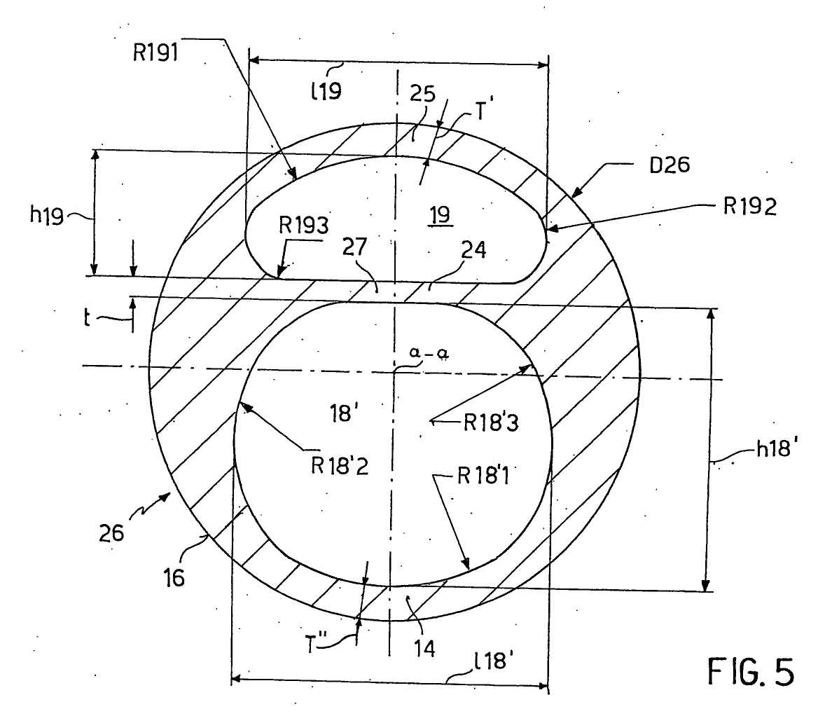

FIG. 5 is a transverse section through an extruded duct suitable for the production of a balloon structure, shown prior to its deformation,

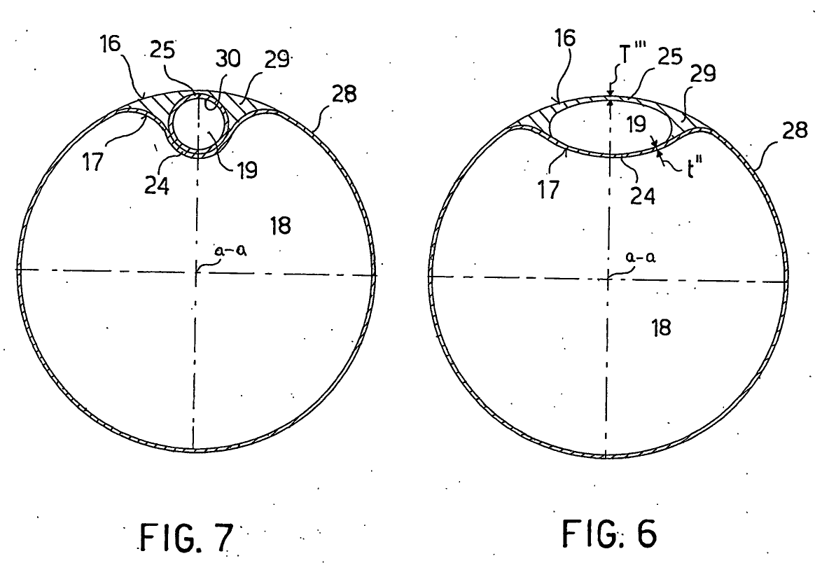

FIGS. 6, 7 and 8 show three transverse sections through ducts coextruded in several materials, shown after their deformation to form balloon structures,

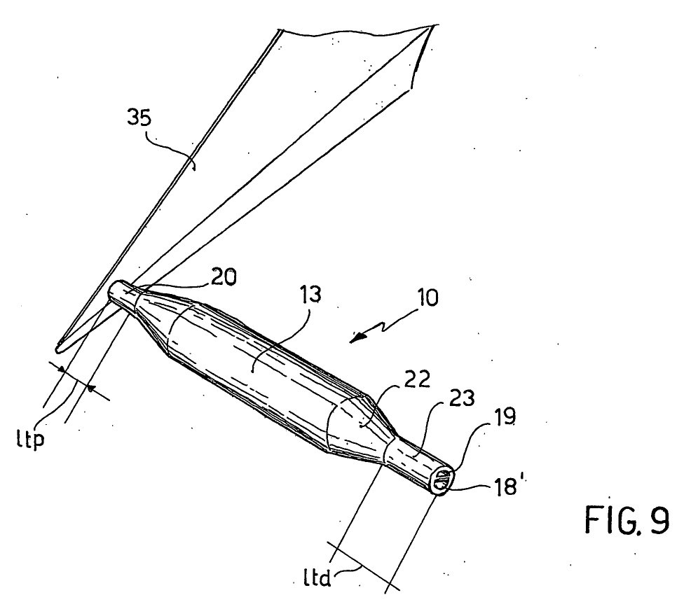

FIG. 9 is a perspective view of a step in the production of a balloon structure,

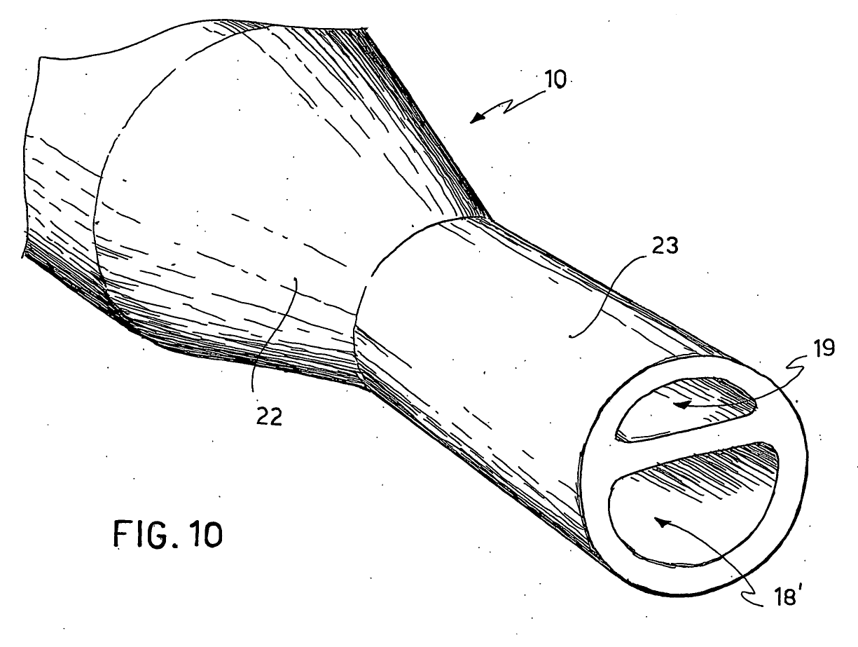

FIG. 10 is an enlarged detail of the distal portion of the balloon structure of FIG. 9, in which the inflation cavities and a guide-wire wall cavity are shown,

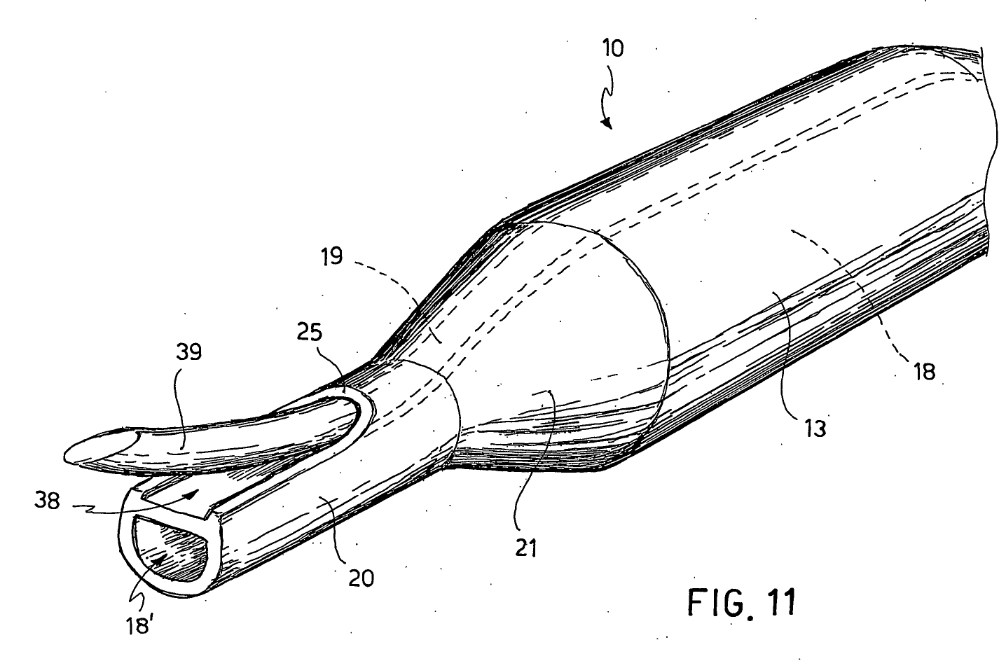

FIG. 11 is a perspective view of a detail of the proximal portion of the balloon structure during a second step of the production of the balloon structure,

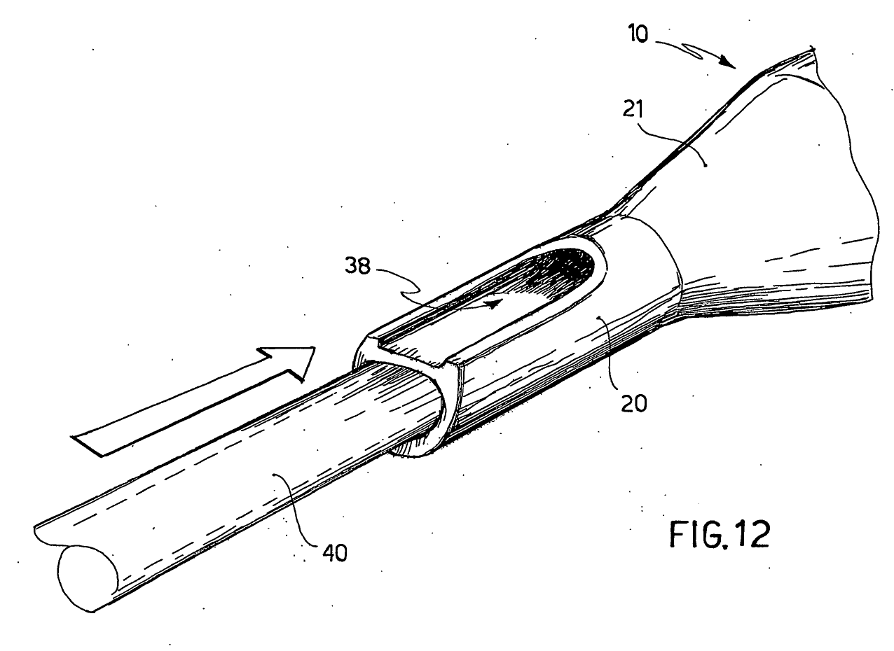

FIG. 12 is a perspective view of a third step in the production of the balloon structure,

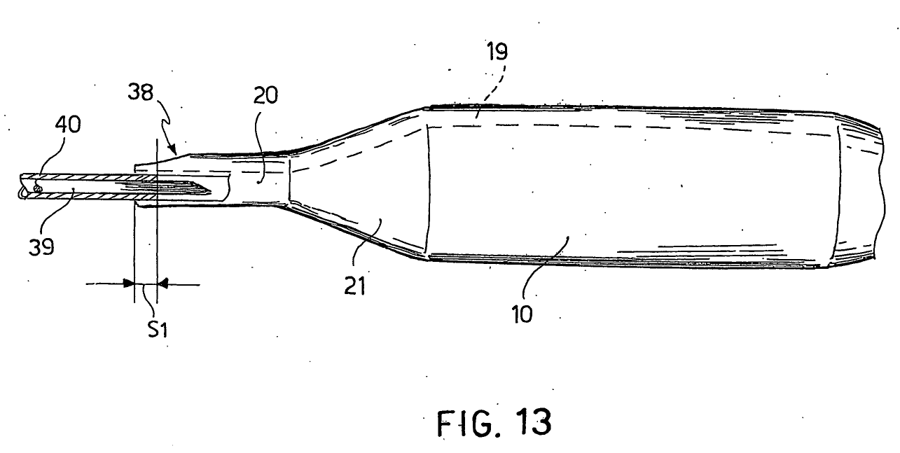

FIG. 13 shows, in longitudinal section, a first step in the production of a catheter comprising a balloon structure,

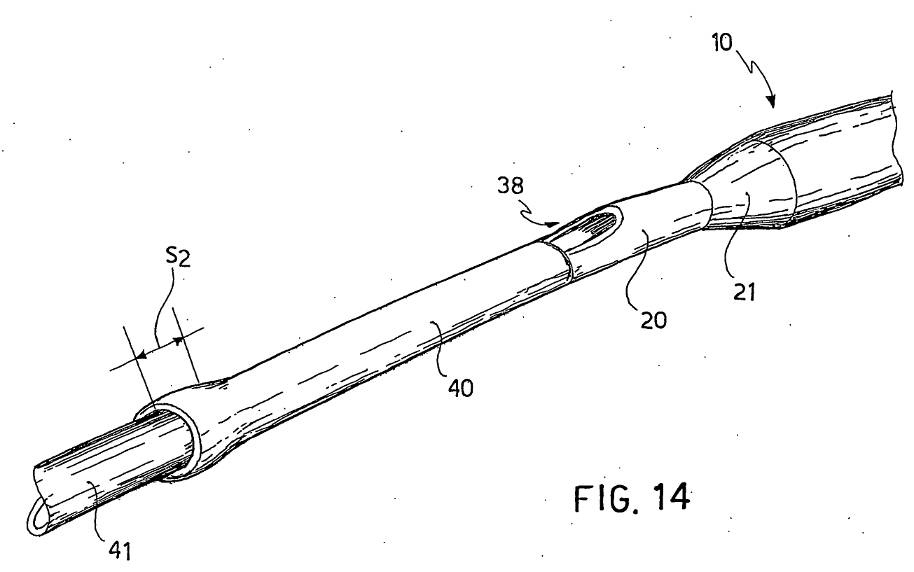

FIG. 14 shows a further step in the production of a shaft of the catheter comprising the balloon structure,

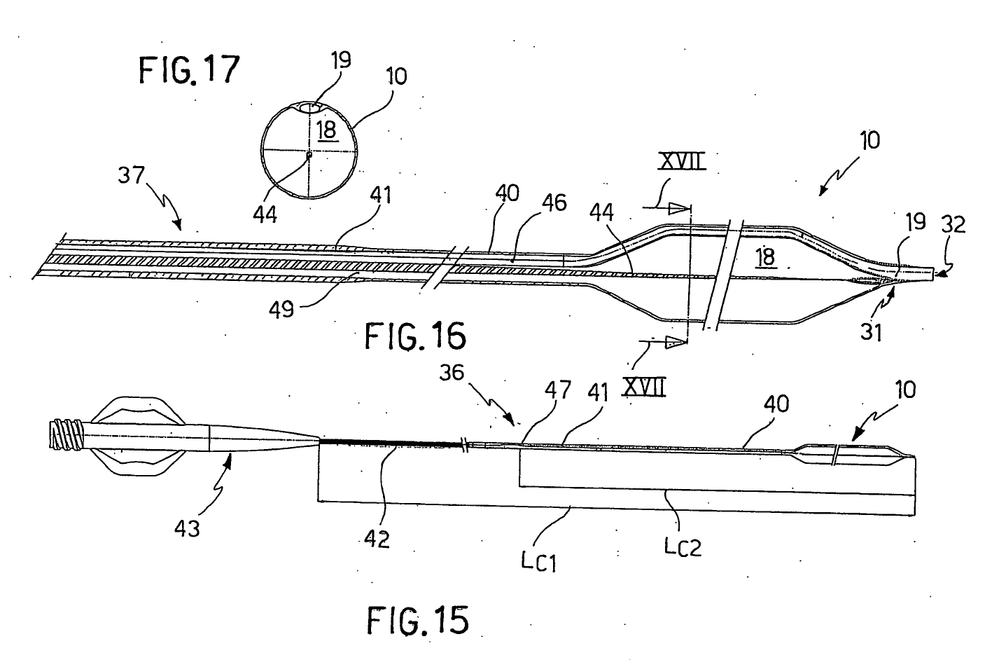

FIG. 15 is a side view of a catheter comprising a balloon structure,

FIG. 16 is a longitudinal section through a detail of the distal portion of the catheter of FIG. 15,

FIG. 17 is a transverse section through the balloon structure of the catheter of FIG. 16, taken on the line XVII-XVII,

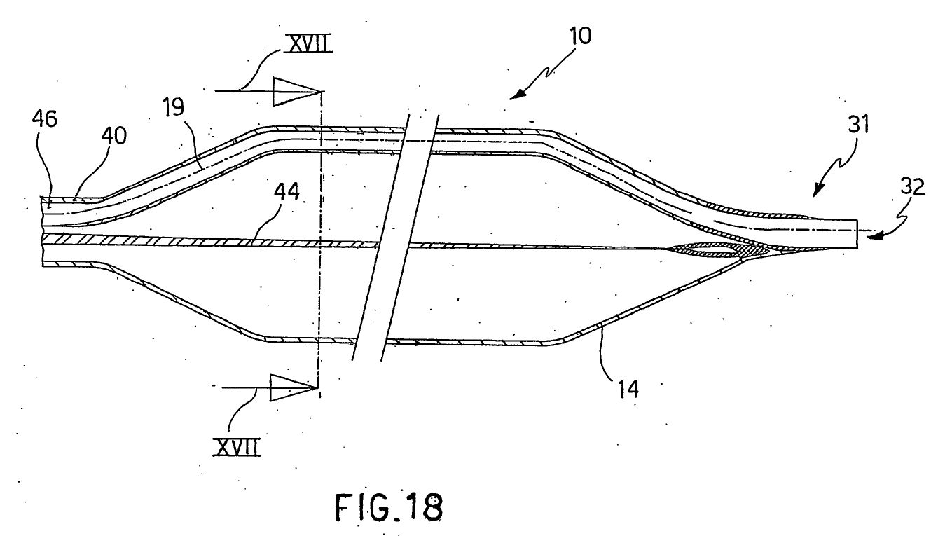

FIG. 18 shows, in longitudinal section, a further detail of the distal portion of the catheter of FIG. 15,

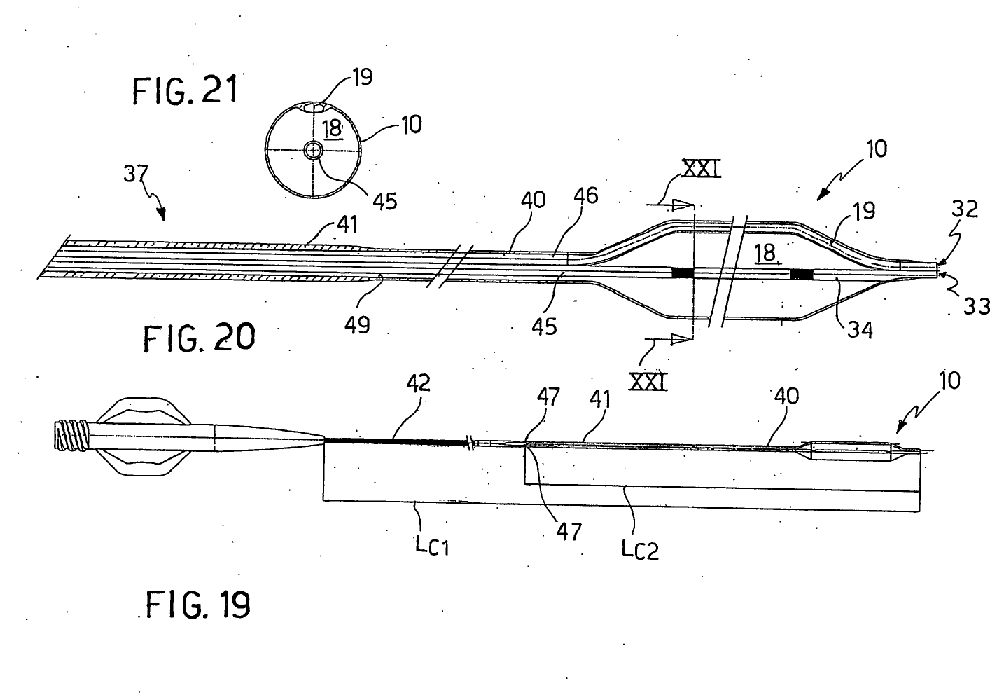

FIG. 19 is a side view of a catheter comprising a further embodiment of the balloon structure,

FIG. 20 is a longitudinal section through a detail of the distal portion of the catheter of FIG. 19,

FIG. 21 is a transverse section through the balloon structure of the catheter of FIG. 20, taken on the line XXI-XXI,

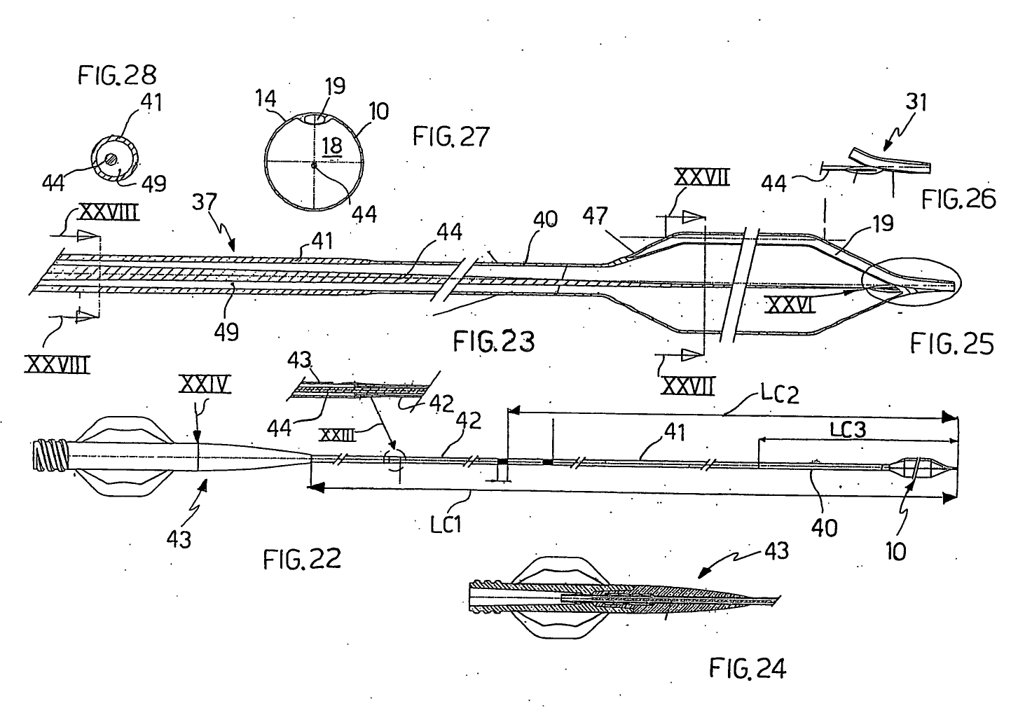

FIG. 22 is a side view of a catheter comprising yet another embodiment of the balloon structure,

FIG. 23 shows, in section, a detail of the shaft of the catheter of FIG. 22, indicated by the arrow XXIII,

FIG. 24 is a section through a proximal connector of the catheter of FIG. 22, indicated by the arrow XXIV,

FIG. 25 is a longitudinal section through a detail of the distal portion of the catheter of FIG. 22,

FIG. 26 shows, in longitudinal section, the tip of the catheter of FIG. 25, indicated by the arrow XXVI in that drawing,

FIG. 27 is a transverse section through the balloon structure of the catheter of FIG. 25, taken on the line XXVII-XXVII,

FIG. 28 is a transverse section through the balloon structure of the catheter of FIG. 25, taken on the line XXVIII-XXVIII,

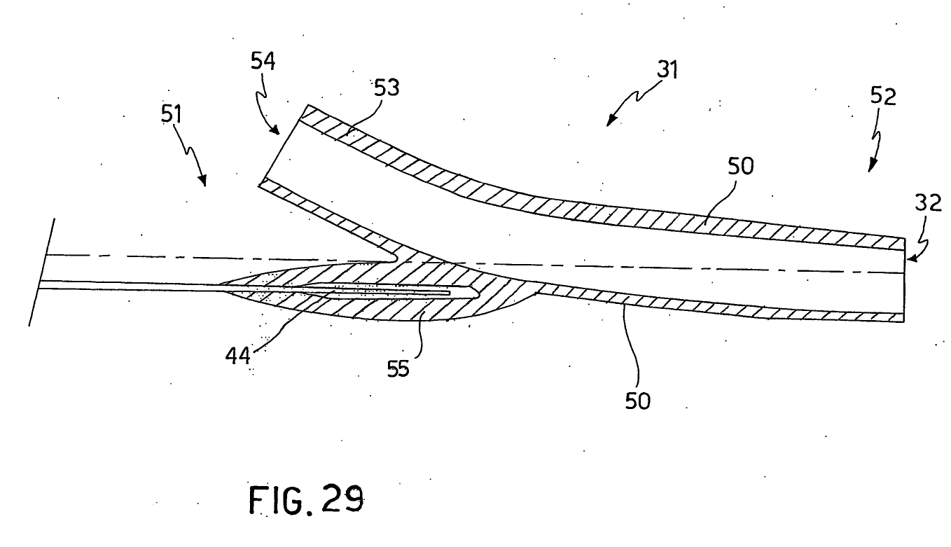

FIG. 29 is a longitudinal section through a tip for a balloon structure,

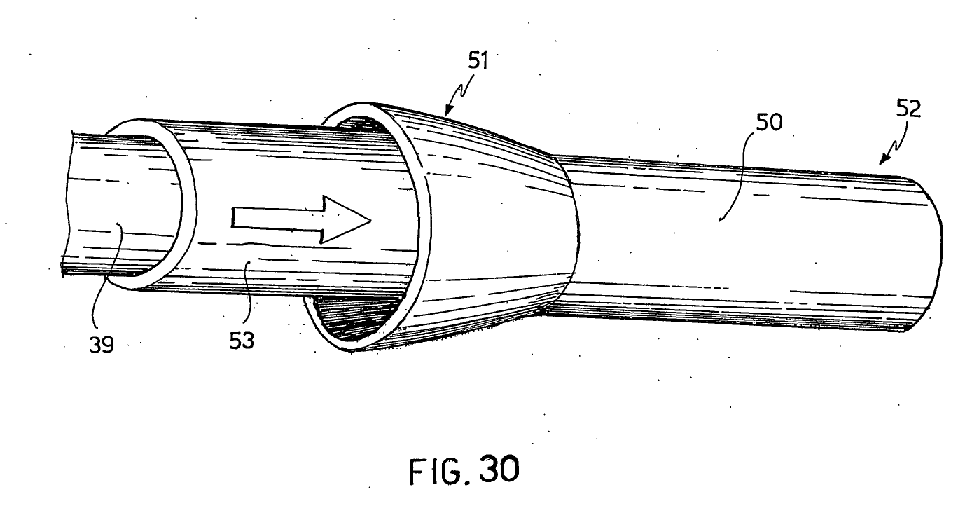

FIG. 30 shows a first step in the production of a tip for a balloon structure,

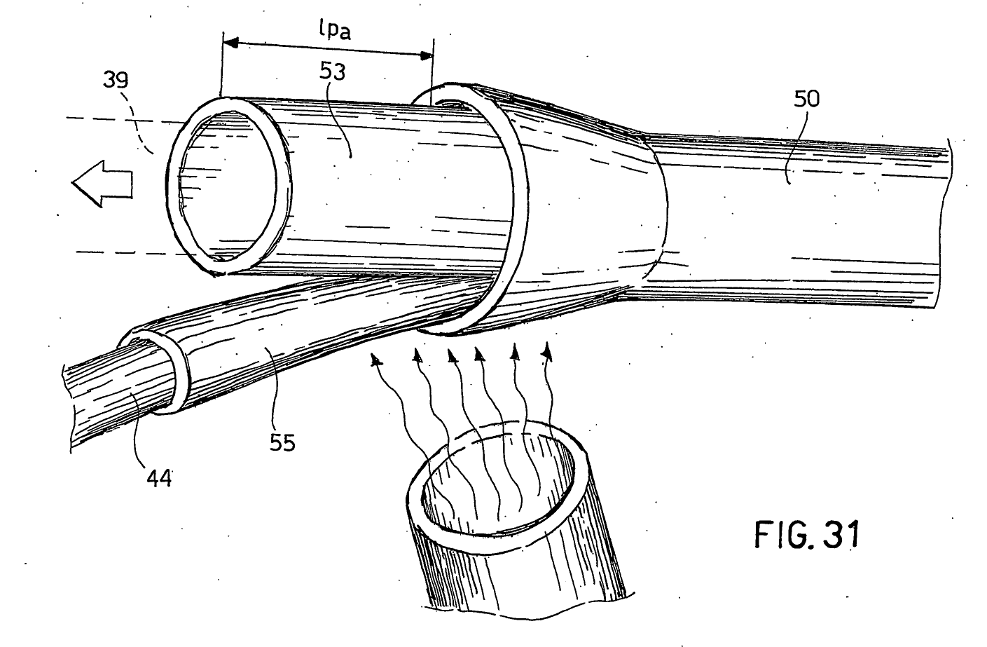

FIG. 31 shows a second step in the production of a tip for a balloon structure,

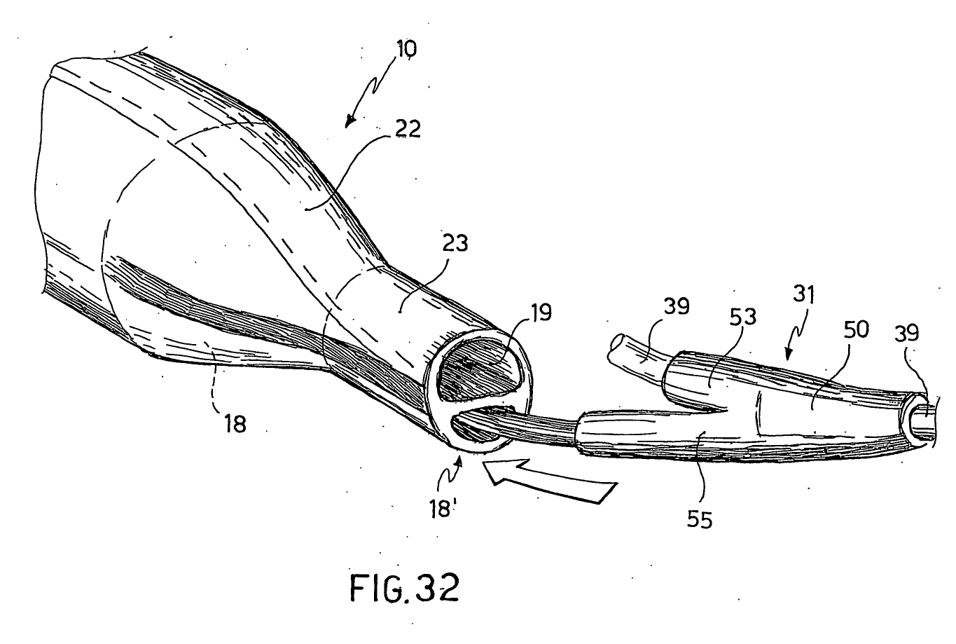

FIG. 32 shows a first step in the mounting of a balloon-structure tip on a distal portion of a balloon structure,

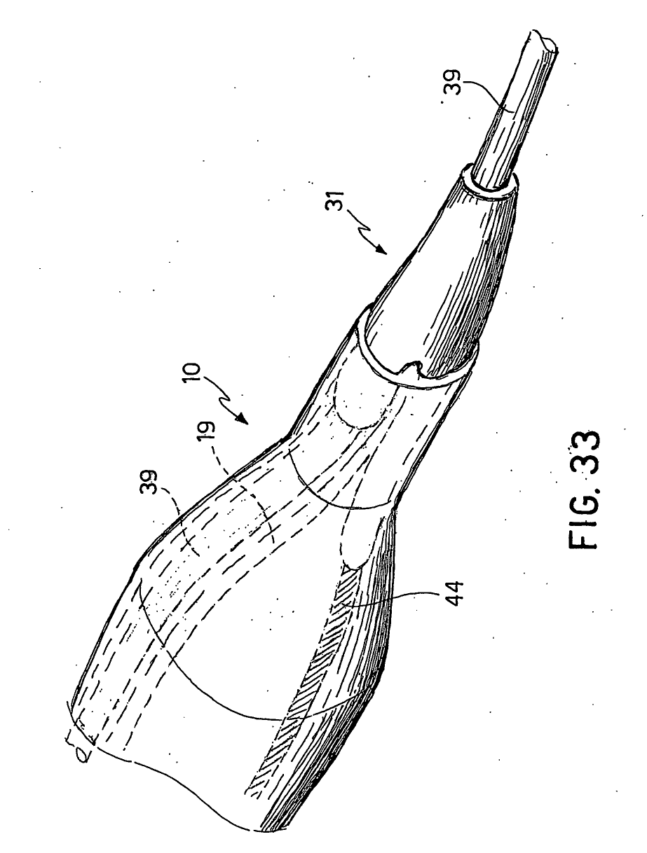

FIG. 33 shows a second step in the mounting of a balloon-structure tip on a distal portion of a balloon structure,

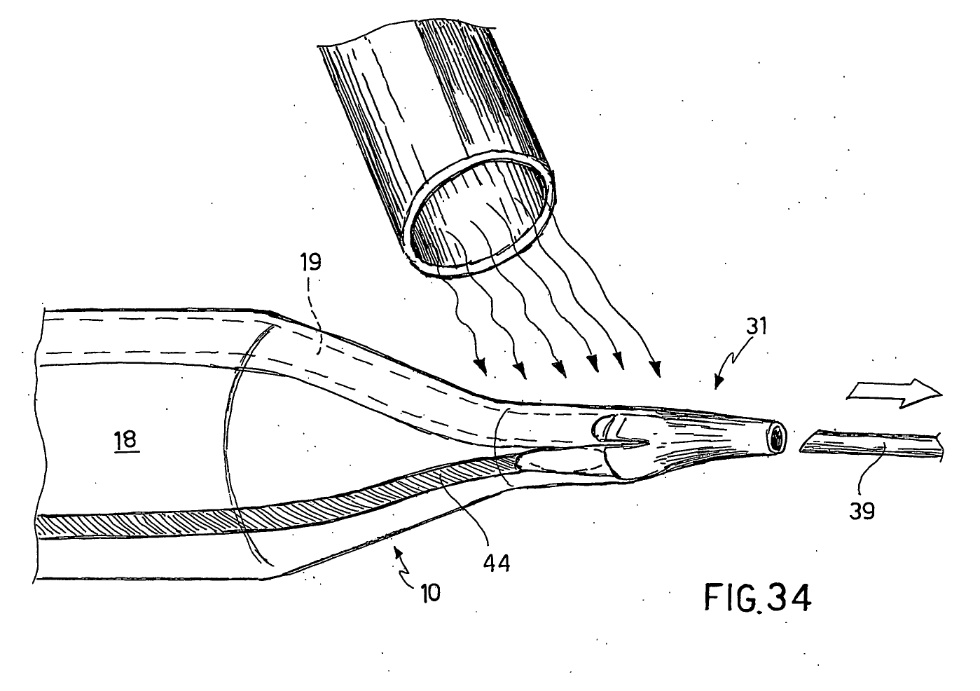

FIG. 34 shows a third step in the mounting of a balloon-structure tip on a distal portion of a balloon structure.

With reference to the above-mentioned drawings, a balloon structure is generally indicated 10.

The balloon structure is used in particular, but in non-limiting manner, as a part included in a catheter, for example, but in non-limiting manner, a catheter for angioplasty or a catheter for depositing an endolumenal prosthesis or stent in a duct, for example, a vascular duct, for example, a coronary or peripheral duct.

The term “balloon structure” is intended to define herein a structure which comprises an inflatable or expandable balloon which can change from a contracted condition, when it is deflated, to an expanded condition when it is inflated with a fluid. During its expansion, the balloon dilates, for example, the walls of a duct or, purely by way of indication, displaces or shifts the plaque of a stenosis which is partially or completely obstructing a lumen of a vessel, obstructing the free flow of blood. The balloon structure can, for example, also be used to expand endolumenal prostheses or stents so as to bring the stents from a condition in which they are contracted onto or clinging to the contracted balloon to an expanded condition for supporting the walls of a portion of a vessel, for example, a blood vessel and for keeping the walls stretched.

In a general embodiment, the balloon structure 10 is of predominantly longitudinal extent, for example, it is a structure which extends about a longitudinal axis a-a. The structure has a proximal end 11, a distal end 12, and an intermediate portion 13 of predefined longitudinal extent L, suitable for performing an expansion in an object to be dilated, for example, the walls of a duct or an endolumenal prosthesis which initially clings to the intermediate portion in a contracted condition (FIG. 1).

The balloon structure 10 comprises a wall 14 which has, at any point, an annular cross-section 15 (FIG. 2) transverse the longitudinal axis a-a. The annular cross-section 15 is delimited externally by an outer surface 16 which, at least in the intermediate portion 13, is suitable for coming into contact with the object to be dilated. The section 15 is delimited internally by an inner surface 17 which delimits an inflation chamber 18.

At least one wall lumen or cavity 19 is advantageously provided in the wall 14. This wall cavity is formed within the annular cross-section 15 which delimits the inflation chamber 18 so as to be disposed between the outer surface 16 and the inner surface 17.

The wall cavity extends longitudinally relative to the balloon structure 10, without interruption, between the proximal end 11 and the distal end 12 so that, when the balloon structure is inflated or expanded, the outer surface 16 of the intermediate portion 13 has uniform curvature around the entire annular extent of the cross-section 15 in a section transverse the longitudinal extent of the balloon structure 10 (FIGS. 1 and 2).

The expression “extends without interruption” is intended mainly but not exclusively to mean without diverting partitions and/or openings, for example, lateral apertures facing from the wall cavity 19 towards the inflation chamber 18 or lateral apertures which open in the outer surface 16 of the active portion L of the balloon structure 10, for example, the intermediate portion 13. As can be seen, for example, from FIGS. 1 and 2, by virtue of the wall cavity disposed in the wall of the balloon structure, the inflation chamber 18 is free of any impediment to a free flow of fluid into and out of the chamber. Moreover, the balloon structure 10 does not have on the outer surface 16 any obstacles to its free rotation, for example, to the rotation or twisting of a catheter comprising the balloon structure about itself in a narrow vessel.

According to a first embodiment, when the balloon structure is inflated or expanded, the outer surface 16 of the intermediate portion 13 is free of protuberances or recesses such as, for example, protuberances created by a duct associated with the. annular section externally to form an overall “8”-shaped cross-section. Instead, when the inflation chamber 18 is expanded, the balloon structure 10 advantageously has the outer surface 16 which has an annular cross-section transverse the longitudinal extent of the annular balloon structure, therefore said outer surface is suitable for applying an uniform pressure all around. The outer surface 16 according to the present invention is also free of recesses such as a pocket or channel formed, for example, by a fold in the balloon wall. Such protuberances or recesses would create obstacles to the free twisting of a catheter comprising the balloon structure. Instead, by virtue of an outer surface which is free of protuberances or recesses, when the balloon structure is in an expanded condition, its angular position can be modified freely relative to its longitudinal axis a-a when it is housed in a duct such as a blood vessel.

According to a further embodiment, the wall cavity 19 is within the wall 14 which delimits the inflation chamber 19 for the whole of its extent that affects the balloon structure (FIGS. 3 and 4).

When the balloon structure is inflated or expanded, the outer surface 16 of the intermediate portion 13 is advantageously cylindrical.

Preferably, when the inflation chamber 18 is expanded, the balloon structure 10 has an outer surface 16 of substantially circular cross-section transverse the longitudinal extent of the balloon structure.

According to one embodiment, the balloon structure 10 comprises a proximal tubular portion 20 in the vicinity of the proximal end 11.

The balloon structure also comprises a proximal shank 21 which connects the proximal tubular portion 20 to the intermediate portion 13. The proximal shank 21 advantageously has an internal taper angle “a” of between 20 degrees. and 40 degrees. According to a further embodiment, the shank 21 has an internal taper angle of 30 degrees (FIG. 1).

According to one embodiment, the balloon structure 10 comprises a distal connecting shank 22 between the intermediate portion 13 and a portion 23 for connection to a distal catheter tip, as will be described in detail below. The distal shank 22 advantageously has an internal taper angle “b” of between 20 degrees and 40 degrees, preferably 30 degrees.

The wall cavity 19 is preferably separated from the inflation chamber 18 by an internal portion 24 of the wall 14 (FIGS. 2 and 4). The cavity 19 is separated from the outer surface 16 by an external portion 25 of the wall 14.

According to one embodiment, when the balloon structure is inflated or expanded, the internal surface 17 of the intermediate portion 13 is smoothed, rounded, or free of sharp corners. When the balloon structure is inflated or expanded, the inner surface 17 of the intermediate portion 13 advantageously has an annular cross-section transverse the longitudinal extent of the balloon structure.

According to one embodiment, the balloon structure 10 is produced from an extruded tube or duct, which is generally indicated 26 when it is not yet processed in order to become a balloon structure, and has at least two cavities 18′ and 19, one of which, for example, the cavity 18′, is subsequently deformed to form the inflation chamber 18 of the balloon structure 10 and is hereinafter also defined as the inflation cavity 18′ (FIG. 5).

Prior to the deformation of a cavity 18′ thereof to form an inflation chamber 18, the extruded tube 26 advantageously has an at least partially flat partition 27 separating the at least two cavities. After the deformation of a cavity 18′ to form an inflation chamber 18, the separating partition 27 will advantageously constitute an internal portion 24 of the wall which separates the wall cavity 19 from the inflation chamber 18.

According to one embodiment, prior to the deformation of a cavity 18′ of the extruded tube 26 to form an inflation chamber 18, the extruded tube 26 has a partition 27 for separating the at least two cavities 19 and 18′; this partition has, in a cross-section transverse the extruded tube, a minimum thickness t of between 55% and 100% of the minimum thickness T′ or T″ of the wall portion 14 or 25 which separates one of the cavities 18′ and 19 from the outer surface 16. Advantageously, prior to the deformation of a cavity 18′ of the extruded tube 26 to form an inflation chamber 18, the extruded tube 26 has a partition 27 for separating the at least two cavities 18′ and 19; this partition has, in cross-section transverse the extruded tube, a minimum thickness t of between 60% and 70% of the minimum thickness T′ or T″ of the wall portion which separates one of the cavities 18′ and 19 from the outer surface 26 (FIG. 5).

According to one embodiment, a cavity 19 in the extruded tube 26 has a predefined width l19 and a predefined height h19. Advantageously, the cavity 19 has walls that are connected to one another and are preferably formed, in cross-section, by arcs of predefined radius, that is, a first arc, for example, the arc forming the inner wall of the external wall portion 25 of radius R191 concentric with the axis a-a of the tube 26, and then radii connecting the wall portion 25 to the partially flat partition 27, for example, predefined radii R192 and R193. The second cavity 18′ has a predefined width l18′ and height h18′ and delimiting walls which are formed in cross-section, by arcs of predefined radius, that is, a first arc of radius R18′1, concentric with the axis a-a of the extruded tube 26, and successive arcs with predefined radii R18′2 and R18′3, connected to the first arc and also connected to the partition 27 (FIG. 5).

According to one embodiment, the balloon structure 10 is produced by the expansion of an inflation cavity 18′ of a tube with at least two cavities 18′ and 19, the tube 26 being produced by coextrusion of at least two materials; a first of these materials 28, when expanded, forms the wall 14 or wall portion 24 which delimits the inflation chamber 18 or separates the chamber 18 from the wall cavity 19. Advantageously, the material which delimits the inflation cavity 18′ prior to its deformation and the inflation chamber after its deformation is a semi-compliant or partially yielding material which is resistant to the maximum pressure predefined for the inflation of the balloon structure, such as, for example, nylon or a material with similar performance. With further advantage, a second 29 of these materials forms at least a part of the wall portion 24, 25 which delimits the wall cavity 19. For example, the second material 29 forms the wall portion 25 which separates the wall cavity 19 from the outer surface 16. According to one embodiment, the second material 29 has a greater flexibility than the first material 28 (FIGS. 6 and 7).

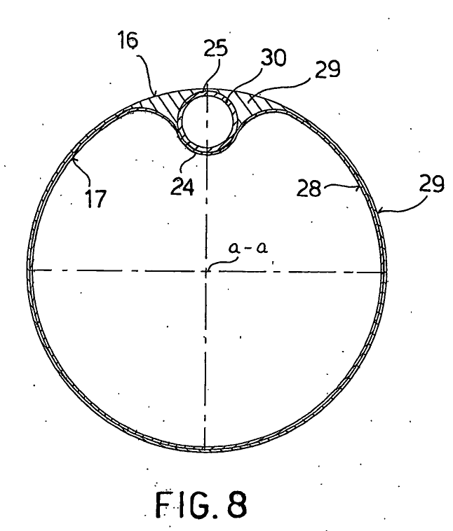

According to a further embodiment, the balloon structure 10 is produced by the expansion of an inflation cavity 18′ of a tube 26 with at least two cavities 18′ and 19, the tube 26 being produced by coextrusion of three materials 28, 29 and 30. Advantageously, the wall cavity 19 is lined or delimited by a layer of material 30 with a coefficient of friction such as to facilitate the sliding of a guide wire housed in the wall cavity 19 (FIGS. 7 and 8).

According to one embodiment, when the balloon structure is inflated or expanded, the wall cavity 19 is separated from the inflation chamber 18 by a wall portion 24 which has, in cross-section transverse the longitudinal extent a-a of the balloon structure, a thickness t″ of between 55% and 100% of the thickness T′″ of a wall portion 25 which separates the wall cavity 19 from the outer surface 16. Preferably, when the balloon structure 10 is inflated or expanded, the wall cavity 19 is separated from the inflation chamber by a wall portion 24 which has, in cross-section transverse the longitudinal extent a-a of the balloon structure 10, a thickness t″ of between 60% and 70% of the thickness T′″ of a wall portion 25 which separates the wall cavity 19 from the outer surface 16. Advantageously the internal wall portion 24 and the external wall portion 25 are of equal minimum thickness (FIG. 6).

According to one embodiment, the inflation chamber 18 is closed in a leaktight manner onto an apex tip 31, leaving only apex access openings 32, 33 to one or more guide-wire cavities 19 and 34 (FIGS. 16 and 20).

According to one embodiment, an inflatable balloon structure for catheters comprises a wall which has, transverse a longitudinal extent thereof, at any point, an annular cross-section delimited externally by an outer surface which, at least in an intermediate portion, is suitable for coming into contact with the object to be expanded, and internally by an inner surface which delimits an inflation chamber. At least one wall cavity is provided in this wall and is formed within the annular cross-section which delimits the inflation chamber so as to be disposed between the outer surface and the inner surface, the cavity extending without interruptions and/or openings, longitudinally relative to the balloon structure, between the proximal end and the distal end.

A method for the production of the balloon structure 10 is described by way of example below.

First of all, a tubular duct 26 of predominant longitudinal extent a-a is provided, the tubular duct 26 having at least two cavities 18′ and 19 which extend along its entire longitudinal extent and are kept separated from one another, for example, by a partition 27 which is preferably integral with the structure of the tubular duct. The at least two cavities 18′ and 19 extend from proximal openings to distal openings provided at the proximal end 11 and at the distal end 12 of the duct, respectively.

At least a portion of the duct is then inserted in a die provided with a cavity that is widened to form the desired shape of the expanded balloon and at least one of the distal or proximal openings of the duct is closed. In particular, one of the openings of a cavity 18′ which is intended to form the inflation chamber 18 of the balloon structure is closed in a leaktight manner. Alternatively, the cavity or both cavities may be closed in a leaktight manner by obstructing or choking a portion of the duct which is disposed outside the die.

The portion of duct that is disposed in the die is then heated to a temperature which permits permanent deformation of the material or of one of the materials of which the tubular duct is made. For example, if the duct is made of polyamide and/or nylon and/or Pebax™ and/or high-density polyethylene, with characteristics of a low coefficient of friction, or similar or equivalent materials, used alone or in combination by producing the duct by coextrusion, the die may be heated to a temperature T of between 80° centigrade and 120° centigrade, preferably 100° centigrade.

At this point, fluid can be admitted under pressure to one of the two cavities, that is, the cavity 18′ which will form the inflation chamber 18, so as to deform the wall 14 of the duct which delimits this cavity, causing it to fit against the walls of the widened cavity delimited by the die, meanwhile, for example, the duct is stretched. If fluid under pressure is admitted solely to the cavity 18′ which will form the inflation chamber, the second cavity 19 remains incorporated in the wall thus deformed which delimits the inflation chamber 18. In particular, if, for example, the die is shaped so as to delimit a cylindrical cavity in order to produce a balloon with a cylindrical outer surface, the cavity to which the pressure is admitted deforms in a manner such that the wall 14 which delimits its inflation chamber will have a cylindrical outer surface 16 and the second cavity or wall cavity 19 will deform, remaining incorporated within the wall.

According to one embodiment, to prevent the second cavity 19 from being closed because of the deformation of the first cavity 18′ to form an inflation chamber 18, before fluid is admitted to one of the cavities 18′ under pressure, a stylet 39 is inserted in the other cavity. This prevents the deformation of the heated wall from blocking this other cavity 19.

According to a further embodiment, the stylet is covered with non-stick material such as Teflon™ which enables the stylet to be removed from the cavity or wall cavity 19 after the deformation of the inflation chamber 18 at high temperature, without the wall portion 24 which delimits the wall cavity 19 being welded to the stylet or to other wall portions 25, blocking the cavity or preventing removal of the stylet.

As a result of the deformation of the duct with two cavities in the die, a balloon structure 10, for example, as described above, is obtained.

Further variants may also be provided. For example, it is possible to use, initially, a duct with three or more cavities in which only one of these cavities is deformed at high temperature to form the inflation chamber as described above, so as to leave the other two or more cavities incorporated in the wall of the balloon, for example, in predetermined positions. With reference to a cross-section transverse the longitudinal extent of the duct or of the balloon structure thus obtained, the cavities incorporated in the wall of the balloon may be spaced apart angularly by 180 degrees, that is on opposite sides of the cross-section with respect to the inflation chamber, or may be spaced apart by angles of 120 degrees, or even 90 degrees.

The description relating to a possible embodiment of a catheter is given below. First of all, the balloon structure 10, produced as described above, is cut with a tool 35 in a proximal region 11 relative to the balloon so as to leave, for example, from the balloon towards the proximal end, a tubular portion 20 which has a length lt which varies from 1 mm to 150 mm. Preferably, the balloon structure 10 is cut proximally at about 1.5 mm-2.5 mm from the balloon or from the proximal transition region or shank 21, which transition region is provided between the intermediate portion 13 of maximum extent of the balloon and a proximal tubular portion 20. In other words, the proximal tubular portion 20 is cut so that it is about 1.5 mm-2.5 mm long. This is performed, for example, so as to modify the characteristics of the catheter 36 which are to be obtained immediately in front of or proximally relative to the balloon. It is in fact preferable, for example, to have different characteristics for the portion of the catheter in which the balloon is provided, in comparison with the portion of the catheter in which the shaft 37 or the thrust body is provided. The tip 31 and the balloon structure 10 of the catheter are preferably made of softer and more flexible material than the shaft 37 which has to exert the necessary thrust on the balloon structure 10 for it to be inserted in the desired duct (FIGS. 9 and 10).

With reference to the die for producing the balloon structure, this can be shaped in a manner such as to delimit a cavity for producing a cylindrical or oval balloon with two connecting portions or sections or connecting shanks 21 and 22 between the intermediate portion or section 13, which is, for example, cylindrical, and the proximal and distal tubular portions or sections 20 and 23. These shanks 21 an 22 are preferably frustoconical. The shanks have, for example, a taper variable from 20 degrees to 40 degrees and are preferably identical for the proximal shank and for the distal shank.

According to one embodiment, a proximal aperture 38 or lateral proximal aperture of the wall cavity 19 is produced a predetermined distance from the balloon or, for example, from the proximal shank 21, in the direction away from the balloon. For example, the lateral proximal aperture 38 may be produced by removing a section of the wall portion 25 which separates the wall cavity 19 from the outer surface 16. For example, this operation is performed with the insertion of a stylet 39 which protects the wall portion 24 that separates the wall cavity 19 from the inflation chamber 18′.

A proximal tube is then connected or joined to the balloon structure 10, forming the shaft 37 of the catheter 36. First of all, a not adhesive, such as PTFE, coated stylet or a Teflon™-coated stylet is fitted in the inflation cavity 18′ which, as described above, is formed by the portion of the cavity which is in the proximal tubular portion 20 and which has not been transformed into the inflation chamber 18, and a tube or distal section of the shaft 40, for example, a single-cavity duct or a duct with solely an inflation chamber 18′, is fitted thereon (FIGS. 12 and 13).

The distal section of the shaft 40 will preferably be inserted in the proximal tubular portion 20 for a distance which ensures leaktightness and sufficient structural strength and which at the same time does not stiffen the shaft 37 locally, for example, the distal section of the shaft 40 is inserted for a distance of 2 times the thickness of the tubular portion to be welded, such as 1 mm-3 mm, preferably a distance of 1.5 mm-2.5 mm. When the overlap has been formed, it is welded, forming a joint, for example, by means of a jet of hot air at between 180° centigrade and 220° centigrade, in any case at a temperature greater than the melting temperature of the used materials, the balloon structure being protected, for example, by a screen. The region to be welded is covered with a film or a tubular piece of a thermo-retractile material which deforms the joint region during the heating of the region, and permits a gradual transfer of heat and which can be removed after the welding, for example, polyefin. To ensure that the inflation cavity 18′ is not blocked during the welding, a Teflon-coated stylet 39 is kept therein.

In order to vary the stiffness and the thrust capacity of the shaft 37 of the catheter 36 along its longitudinal extent, an intermediate shaft section 41, for example stiffer than the distal section 40, may be fixed or joined proximally to this first, distal shaft section. In this case, the inflation cavity 18′ which is also present in the intermediate shaft section 41 is again protected by the insertion of a Teflon-coated stylet 39. The welding is performed by the same procedures as described above. In this case, the shaft sections are again overlapped for a portion of predetermined extent s2 which ensures the necessary structural strength and at the same time the desired flexibility. The joints between the balloon structure and the distal and intermediate shaft sections are illustrated schematically in FIGS. 12, 13 and 14, purely to show their characteristics. After welding, the regions affected, that is the joint regions, will nevertheless have external dimensions approximately equal to those of the larger-dimensioned duct, or even smaller.

An embodiment of an apex tip, for example, as shown in FIG. 29, to be connected or joined to a distal end of a balloon structure, as described above to form an apex aperture for the wall cavity and a leaktight closure of the inflation chamber, is described below.

First of all, an apex tube 50 is provided, the tube 50 having a proximal end 51 and a distal end 52 in which an apex opening 32 is provided (FIG. 30).

Then a proximal portion of the apex tube 50 is widened, for example, by squashing or flaring the tube on opposite sides, rendering the cavity oval locally.

A connecting tube 53 to be inserted in a wall cavity 19 of a balloon structure 10 is then provided; the connecting tube 53 has a cavity suitable for housing a guide-wire.

With the aid of a stylet 39, for example, a Teflon-coated stylet, the distal portion of the connecting tube 53 is fitted in the widened portion of the apex tube 50 so as to form a continuous cavity between a proximal opening 54 of the connecting tube 53 and the apex opening 32 of the apex tube 50.

According to one particular embodiment, an anchoring tube 55 is provided for insertion with a distal portion thereof in the widened apex tube 50 so as to be disposed at least partially beside the connecting tube 53. The anchoring tube 55 is suitable for anchoring a thrust wire 44 and is suitable for the leaktight closure of the distal opening of the inflation chamber 18 of a balloon structure 10 (for example, as shown in FIG. 18). The welding between the apex tube 50, the connecting tube 53, and the anchoring tube 55 is then performed in a manner similar to that described above, taking particular care not to close the cavity 19 by inserting a Teflon-coated stylet 39 (FIG. 31) beforehand. Whilst performing the joining, it is necessary to take care that the connecting tube projects from the apex tube by a predetermined distance lpa which, for example, is variable between 1.5 mm and 2.5 mm, so as to allow the connecting tube 53 to be inserted in the wall cavity 19 whilst avoiding excessive stiffening of the distal portion of the balloon structure and at the same time ensuring the necessary structural strength.

The apex tip 31 is then ready to be connected to a balloon structure. The thrust wire 44 is inserted in the inflation cavity 18′ in the inflation chamber 18, and then in the inflation cavity 18′ of the catheter shaft 37 until the anchoring tube 55 is inserted in a leaktight manner in the distal opening of the inflation cavity 18′ (FIG. 32). During this operation, the connecting tube 53 is simultaneously inserted in the wall cavity 19 (FIG. 33). The apex tip 31 is then welded to the balloon structure by the method described above (FIG. 34), with suitable screening of the balloon structure 10. Finally, the stylet is removed from the wall cavity 19.

According to a further embodiment, a second connecting tube is provided for insertion with a distal portion thereof in the apex tube so as to be disposed at least partially beside the first connecting tube. The second connecting tube is suitable for forming a second guide-wire cavity 34 between a proximal opening of the second connecting tube and the apex opening 33 of the apex tube, and is suitable for the leaktight closure of a distal opening of an inflation chamber of a balloon structure. This second connecting cavity 34 is suitable for connection to a guide-wire duct 45 which is provided inside the catheter (FIG. 20) in one embodiment.

According to one embodiment, a catheter apex tip is thus produced. The tip has a proximal end and an apex end and comprises a tubular apex portion disposed in the vicinity of the apex end, and a proximal connecting tube disposed in the vicinity of the proximal end. The proximal connecting tube is partially housed with a distal portion thereof inside a proximal portion of the apex tube. The connecting tube is connected to the apex tube so as to form a cavity which extends without interruption from an opening disposed at the proximal end of the connecting tube to an opening disposed at the apex end of the apex tube. The connecting tube is suitable for connection to a wall cavity suitable for housing a guide wire, which cavity is provided within a wall that delimits a balloon inflation chamber. The apex tube is fitted proximally and in a leaktight manner in a distal opening of a balloon structure for the leaktight closure thereof.

According to a further embodiment, a catheter tip has a proximal end and an apex end and comprises a tubular apex portion disposed in the vicinity of the apex end, a proximal connecting tube disposed in the vicinity of the proximal end, and a tube for anchoring a thrust wire or rod, also disposed in the vicinity of the proximal end. The anchoring tube and the proximal connecting tube are arranged at least partially side by side and are housed with respective distal portions inside a proximal portion of the apex tube. The connecting tube is connected to the apex tube so as to form a cavity which extends without interruption from an opening disposed at the proximal end of the connecting tube to an opening disposed at the apex end of the apex tube. The connecting tube is suitable for connection to a wall cavity suitable for housing a guide wire, which cavity is provided within a wall that delimits a balloon inflation chamber. The anchoring tube is closed distally and in a leaktight manner and is suitable for connection to a distal opening of a balloon structure for the leaktight closure thereof and for the anchorage of a distal end of a thrust wire. The anchoring tube, the connecting tube, and the apex tube are advantageously welded to form a single body (FIGS. 16, 18, 25, 26 and 29).

According to yet another embodiment, a catheter tip has a proximal end and an apex end and comprises a tubular apex portion disposed in the vicinity of the apex end, a first proximal connecting tube disposed in the vicinity of the proximal end, and. a second connecting tube also disposed in the vicinity of the proximal end. The first connecting tube and the second connecting tube are arranged at least partially side by side and are housed with respective distal portions inside a proximal portion of the apex tube. The first and second connecting tubes are connected to the apex tube so as to form cavities which extend without interruption from an opening disposed at the proximal ends of the connecting tubes to at least one opening disposed at the apex end of the apex tube. The first connecting tube is suitable for connection to a wall cavity suitable for housing a guide wire, which wall cavity is provided within a wall that delimits a balloon inflation chamber. The second connection tube is suitable for connection to a guide-wire duct which is suitable for housing a guide wire and is disposed inside the balloon structure. The first and second connecting tubes and the apex tube are advantageously welded to form a single body (FIG. 20).

The connection of the balloon structure to the shaft and to the apex tip produces a catheter as shown in FIGS. 15 to 28.

According to one embodiment, a catheter 36 comprises an apex tip 31 which has a proximal end and an apex end and comprises a tubular apex portion 50 disposed in the vicinity of the apex end, and a proximal connecting tube 53 disposed in the vicinity of the proximal end. The proximal connecting tube 53 is partially housed with a distal portion thereof inside a proximal portion of the apex tube 50. The connecting tube is connected to the apex tube so as to form a cavity which extends without interruption from an opening disposed at the proximal end of the connecting tube to an opening disposed at the apex end of the apex tube. The connecting tube is connected to a wall cavity 19 suitable for housing a guide wire. The wall cavity is provided within a wall that delimits an inflation chamber 18 of a balloon structure 10.

According to another embodiment, a catheter comprises an apex tip 31 which has a proximal end and an apex end and comprises a tubular apex portion 50 disposed in the vicinity of the apex end, a proximal connecting tube 53 disposed in the vicinity of the proximal end, and a tube 55 for anchoring a thrust wire 44 or rod, also disposed in the vicinity of the proximal end. The anchoring tube 55 and the proximal connecting tube 53 are arranged at least partially side by side and are housed with respective distal portions inside a proximal portion of the apex tube 50. The connecting tube 53 is connected to the apex tube 50 so as to form a cavity which extends without interruption from an opening disposed at the proximal end of the connecting tube to an opening 32 disposed at the apex end of the apex tube. The connecting tube is connected to a wall cavity 19 suitable for housing a guide wire, and the wall cavity is provided within a wall that delimits an inflation chamber 18 of a balloon structure 10. The anchoring tube 55 is connected to a distal opening of a balloon structure for the leaktight closure thereof and for the anchorage of a distal end of a thrust wire 44 provided inside the balloon structure 10. In the catheter, the anchoring tube, the connecting tube, and the apex tube are advantageously welded to form a single body (FIGS. 15, 16, 18 and 22, 25).

According to yet another embodiment, a catheter comprises an apex tip 31 which has a proximal end and an apex end and comprises a tubular apex portion disposed in the vicinity of the apex end, a first proximal connecting tube disposed in the vicinity of the proximal end, and a second connecting tube also disposed in the vicinity of the proximal end. The first connecting tube and the second connecting tube are arranged at least partially side by side and are housed with respective distal portions inside a proximal portion of the apex tube. The first and second connecting tubes are connected to the apex tube so as to form cavities which extend without interruption from respective openings disposed at the proximal ends of the connecting tubes to at least one opening disposed at the apex end of the apex tube. The first connecting tube is connected to a wall cavity suitable for housing a guide wire, which wall cavity is provided within a wall that delimits a balloon inflation chamber. The second connecting tube is connected to a guide-wire duct 45 suitable for housing a guide wire, the guide-wire -duct being disposed inside the balloon structure. The first and second connecting tubes and the apex tube are advantageously welded to form a single body (FIGS. 19, 20 and 21).

According to one embodiment, a catheter comprises a thrust wire 44 disposed inside the catheter shaft 37 that is connected proximally to the balloon (FIGS. 16, 18 and 25).

A catheter advantageously comprises a guide-wire duct 45 which is disposed inside the catheter shaft 37 that is connected proximally to the balloon (FIGS. 20 and 21).

According to one embodiment, in a catheter, the wall portion which separates the wall cavity from the outer surface has an opening which forms a lateral aperture 47 to allow a guide wire to be inserted in the wall cavity 19 or to emerge therefrom (FIG. 25).

According to one embodiment, a catheter comprises a balloon structure 10 connected proximally to a shaft 37 which comprises an inflation cavity 18′ or 49 that is connected to the inflation chamber 18 in a leaktight manner for the flow of a fluid from the shaft to the inflation chamber and vice versa (FIGS. 16, 20 and 25).

According to a further embodiment, a catheter comprises a balloon structure 10 connected proximally to a shaft 37 comprising a guide-wire cavity 46 that is connected to the wall cavity 19 in a leaktight manner for the passage of a guide wire (FIGS. 16 and 20).

According to one embodiment, a catheter comprises a guide-wire cavity 46 disposed in the wall of the shaft 37 and separated from the outer surface of the shaft by a wall portion.

According to yet another embodiment, a catheter comprises a shaft which has an opening in the wall portion which separates the guide-wire cavity from the outer surface, which opening is suitable for the passage of a guide wire.

Advantageously, a catheter comprises a balloon structure 10 connected proximally to a shaft comprising a guide-wire cavity 46 connected to the wall cavity 19 in a leaktight manner for the passage of a guide wire, the guide-wire cavity being provided in a guide-wire duct 40 provided inside the shaft 37 (FIGS. 16 and 20). With further advantage, the duct is connected in a leaktight manner to a lateral aperture 47 provided in the outer wall of the shaft to allow a guide wire to be inserted in or to emerge from the guide-wire cavity of the guide-wire duct.

Preferably, a catheter comprises a shaft 37 which has a plurality of portions formed with a plurality of ducts of predetermined longitudinal extent LC1, LC2 and LC3, relative to the apex end of the catheter. Advantageously, the shaft has a plurality of portions 40, 41 and 42 formed with ducts made of different materials and/or with different thicknesses, or with the insertion of a stiffening inner tube.

According to one embodiment, a catheter comprises a shaft which has an inflation cavity 49 in flow communication with the inflation chamber 18 of the balloon structure and a thrust wire 44 having a distal end and a proximal end. The thrust wire is advantageously inside the inflation cavity 49. The thrust wire preferably extends along the entire length of the shaft. According to one embodiment, the thrust wire 44 is anchored by its distal end to the balloon structure. The thrust wire 44 is advantageously anchored by its distal end to the tip 31 of the catheter (FIGS. 16 and 17).

According to one embodiment, a catheter comprises a thrust wire 44 connected by its proximal end to an inner tube that is present in the shaft.

A description of a method for the use of the catheter according to the present invention is given below, purely by way of example.

First of all, at least one guide wire is inserted in a vessel which is to be operated on. A catheter, provided with the above-described balloon structure, is then fitted on the guide wire by passing the wire through an apex guide-wire aperture and then sliding it through a guide-wire wall cavity which, at least for its section corresponding to the extent of the balloon, is disposed in the balloon wall, and causing the wire to emerge from a proximal aperture, relative to the balloon. The catheter is then inserted in the vessel, travelling along the guide wire until the balloon is disposed in the operation zone.

Advantageously a further step is provided for advancing the catheter on the guide wire by pushing it by means of a thrust wire provided in the catheter body.

It can be appreciated from the foregoing that, by virtue of the provision of a structure as described above, there are no obstructions inside the inflation chamber, so that rapid inflation and deflation of the balloon are facilitated. Alternatively, for a given performance with regard to balloon inflation and deflation speed, it is possible to produce catheters of very limited transverse dimensions which can reach even very small vessels. This catheter has also been found to be unusually flexible and easy to guide even along tortuous paths.

If the presence of a thrust wire reaching as far as the tip of the catheter is required, it is possible to have direct transmission of the control imparted to the catheter from the proximal connecting element 43 to the tip, achieving greater precision and speed of operation.

In contrast with known catheters, it is also possible to provide a thrust wire and a guide-wire duct in the catheter shaft without reducing performance in comparison with known solutions. In other words, the inflation cavity is not reduced excessively even if a thrust wire and a guide-wire duct are provided inside the catheter shaft, so that an excessive reduction in balloon deflation time is in any case avoided. Moreover, the catheter is not too stiff.

The solution proposed herein is also substantially more compact or less bulky than known solutions.

Further advantages of the solutions proposed are:

the balloon structure proposed is inserted with extreme flexibility even into tortuous branches,

in particular, the provision of an outer surface with uniform curvature renders the shape of the balloon structure particularly capable of penetrating small ducts and obstructions, facilitates a uniform distribution of pressure on an object to be dilated, and also facilitates the orientation of the balloon structure angularly relative to its longitudinal axis (rotation or twisting of the catheter),

the fact that the guide-wire cavity is provided in the wall of the balloon structure means that no obstructions are created inside the inflation chamber so that the admission of fluid in order to inflate or expand the balloon, as well as the discharge of the fluid in order to contract the balloon and to release the cavity of the vessel promptly, for example, to permit the flow of blood in a blood vessel, are particularly quick at constant overall dimensions of the catheter,

with the solution proposed, it is possible to lead the thrust wire and the guide-wire or guide-cable cavity to the catheter tip simultaneously,

with the solution proposed, it is possible to guide the guide-wire in a guide-cable cavity having an extension equal or lower than the balloon structure extension, in order to reduce the friction force exerted by the guide-wire on the guide-cable cavity,

according to some embodiments, it is possible to maintain or to increase the capacity of the inflation cavity or, for a given inflation cavity, it is possible to reduce the transverse dimensions of the catheter, enabling thrust wires of smaller cross-section to be used for a given catheter stiffness, improving the ease of handling of the catheter and its ability to pass through obstructions, as well as reducing friction during the advance of the catheter in the vessel.

Naturally, variants and/or additions may be provided for the embodiments described and illustrated above.

In order to satisfy contingent and specific requirements, a person skilled in the art will be able to apply to the above-described preferred embodiment of the balloon structure and catheter many modifications, adaptations and replacements of elements with other functionally equivalent elements without, however, departing from the scope of the appended claims.

1-71. (canceled)

72. An inflatable balloon structure for catheters, such as a catheter for angioplasty or for depositing an endolumenal prosthesis or stent in a duct, for example, a vascular duct, the balloon structure being of predominant longitudinal extent with a proximal end and a distal end, and being suitable for performing an expansion in an object to be dilated,
the balloon structure comprising a wall which has, transverse the longitudinal extent, at any point, an annular cross-section delimited externally by an outer surface which, at least in an intermediate portion thereof, is suitable for coming into contact with the object to be dilated, and internally by an inner surface which delimits an inflation chamber, in which:
at least one wall cavity is provided in the wall and is formed within the annular cross-section which delimits the inflation chamber so as to be disposed between the outer surface and the inner surface
the cavity extending, without interruptions and/or openings, longitudinally relative to the balloon structure between the proximal end and the distal end so that, when the balloon structure is inflated or expanded, the outer surface of the intermediate portion has, in cross-section transverse the longitudinal extent of the balloon structure, uniform curvature around the entire annular extent of the cross-section.

73. A balloon structure according to claim 72 in which, when the balloon structure is inflated or expanded, the outer surface of the intermediate portion is free of protuberances or recesses.

74. A balloon structure according to claim 72 in which the wall cavity is within the wall which delimits the inflation chamber for the whole of its extent which affects the balloon structure.

75. A balloon structure according to claim 72 which, when the balloon structure is inflated or expanded, the outer surface of the intermediate portion is cylindrical.

76. A balloon structure according to claim 72 in which, when the inflation chamber is expanded, the balloon structure has an annular cross-section of the outer surface, transverse the longitudinal extent of the balloon structure.

77. A balloon structure according to claim 72 in which, when the inflation chamber is expanded, the balloon structure has a substantially circular cross-section of the outer surface, transverse the longitudinal extent of the balloon structure.

78. A balloon structure according to claim 72 in which the balloon comprises a proximal tubular portion in the vicinity of the proximal end.

79. A balloon structure according to claim 72 in which the balloon comprises a proximal shank connecting the proximal tubular portion and an intermediate portion.

80. A balloon structure according to claim 79, in which the proximal shank has an internal taper angle of between 20 degrees and 40 degrees, preferably of 30 degrees.

81. A balloon structure according to claim 72 in which the balloon comprises a distal connecting shank between the intermediate portion and a portion for connection to a distal catheter tip.

82. A balloon structure according to claim 81, in which the distal shank has an internal taper angle of between 20 degrees and 40 degrees, preferably of 30 degrees.

83. A balloon structure according to claim 72, in which the wall cavity is separated from the inflation chamber by an internal portion of the wall.

84. A balloon structure according to claim 72, in which the cavity is separated from the outer surface by an external portion of the wall.

85. A balloon structure according to claim 72, in which, when the balloon structure is inflated or expanded, the inner surface of the intermediate portion is smoothed, rounded, or free of sharp corners.

86. A balloon structure according to claim 72, in which, when the balloon structure is inflated or expanded, the inner surface of the intermediate portion has an annular cross-section, transverse the longitudinal extent of the balloon.

87. A balloon structure according to claim 72, in which the structure is produced from an extruded tube having at least two cavities, one of which is deformed to form the inflation chamber of the balloon structure.

88. A balloon structure according to claim 87, in which, prior to the deformation of a cavity of the extruded tube to form an inflation chamber, the extruded tube has an at least partially flat partition separating the at least two cavities.

89. A balloon structure according to claim 87, in which, prior to the deformation of a cavity of the extruded tube to form an inflation chamber, the extruded tube has a partition separating the at least two cavities, which partition has, in cross-section transverse the extruded tube, a minimum thickness of between 55% and 100% of the minimum thickness of the wall portion which separates one of the cavities from the outer surface.

90. A balloon structure according to claim 87, in which, prior to the deformation of a cavity of the extruded tube to form an inflation chamber, the extruded tube has a partition separating the at least two cavities which partition has, in cross-section transverse the extruded tube, a minimum thickness of between 60% and 70% of the minimum thickness of the wall portion which separates one of the cavities from the outer surface.

91. A balloon structure according to claim 72, in which the balloon structure is produced by the expansion of an inflation cavity of a tube with at least two cavities, the tube being produced by coextrusion of at least two materials, a first of these materials forming the wall or wall portion which delimits the inflation cavity.

92. A balloon structure according to claim 91, in which the material which delimits the inflation cavity is a material that is semi-compliant or partially yielding but resistant to the maximum balloon-inflation pressure.

93. A balloon structure according to claim 72, in which the balloon structure is produced by the expansion of an inflation cavity of a tube with at least two cavities, the tube being produced by coextrusion of at least two materials, a second of these materials forming at least a part of the wall portion which delimits a wall cavity.

94. A balloon structure according to claim 93, in which the second material forms the wall portion which separates the wall cavity from the outer surface.

95. A balloon structure according to claim 93, in which the second material has a greater flexibility than the first material.

96. A balloon structure according to claim 72, in which the wall cavity is coated with or delimited by a layer of material with a coefficient of friction such as to facilitate the sliding of a guide wire housed in the wall cavity.

97. A balloon structure according to claim 72, in which the balloon structure is produced by the expansion of an inflation cavity of a tube with at least two cavities, the tube being produced by coextrusion of three materials.

98. A balloon structure according to claim 72, in which, when the balloon structure is inflated or expanded, the wall cavity is separated from the inflation chamber by a wall portion which has, in cross-section transverse the longitudinal extent of the balloon, a thickness of between 55% and 100% of the thickness of a wall portion which separates the wall cavity from the outer surface.

99. A balloon structure according to claim 72, in which, when the balloon structure is inflated or expanded, the wall cavity is separated from the inflation chamber by a wall portion which has, in cross-section transverse the longitudinal extent of the balloon, a thickness of between 60% and 70% of the thickness of a wall portion which separates the wall cavity from the outer surface.

100. A balloon structure according to claim 72, in which the inflation chamber is closed in a leaktight manner onto an apex tip, leaving solely openings for access to one or more guide-wire cavities.

101. An inflatable balloon structure for catheters, such as a catheter for angioplasty or for depositing an endolumenal prosthesis or stent in a duct, for example, a vascular duct, the balloon structure being of predominant longitudinal extent with a proximal end and a distal end and being suitable for performing an expansion in an object to be dilated, the balloon structure comprising a wall which has, transverse the longitudinal extent, at any point, an annular cross-section delimited externally by an outer surface which, at least in an intermediate portion, is suitable for coming into contact with the object to be dilated, and internally by an inner surface which delimits an inflation chamber, in which at least one wall cavity is provided in the wall and is formed within the annular cross-section which delimits the inflation chamber, so as to be disposed between the outer surface and the inner surface, the cavity extending without interruptions and/or openings, longitudinally relative to the balloon structure, between the proximal end and the distal end.

102. A method of producing a balloon structure
said balloon structure being of predominant longitudinal extent with a proximal end and a distal end, and being suitable for performing an expansion in an object to be dilated, the balloon structure comprising a wall which has, transverse the longitudinal extent, at any point, an annular cross-section delimited externally by an outer surface which, at least in an intermediate portion thereof, is suitable for coming into contact with the object to be dilated, and internally by an inner surface which delimits an inflation chamber, in which:
at least one wall cavity is provided in the wall and is formed within the annular cross-section which delimits the inflation chamber so as to be disposed between the outer surface and the inner surface
the cavity extending, without interruptions and/or openings, longitudinally relative to the balloon structure between the proximal end and the distal end so that, when the balloon structure is inflated or expanded, the outer surface of the intermediate portion has, in cross-section transverse the longitudinal extent of the balloon structure, uniform curvature around the entire annular extent of the cross-section;
said method provides for the steps of:
providing a tubular duct of predominant longitudinal extent, provided with at least two cavities which extend along its entire longitudinal extent whilst remaining separate from one another, between proximal openings and distal openings,
inserting at least a portion of the duct in a die provided with a cavity that is widened to form the desired shape of the expanded balloon,
closing in a leaktight manner one of the distal and proximal openings, or sections of the duct outside the die, of at least one cavity to be expanded,
heating the portion of duct that is disposed in the die to a temperature which permits permanent deformation of the material or of one of the materials which constitute the tubular duct, at least in the region of the cavity to be expanded,
admitting fluid under pressure to the cavity to be expanded so as to deform the wall of the duct which delimits the cavity, causing the wall to fit against the walls of the widened cavity delimited by the die, the second cavity remaining incorporated in the wall thus deformed.

103. A method according to claim 102, in which, before the admission of fluid under pressure to one of the cavities, a stylet is inserted in the other cavity so as to prevent the deformation of the heated wall from obstructing this other cavity.

104. A method according to claim 103, in which the stylet is coated with non-stick material such as, for example, Teflon™.

105. A balloon catheter comprising a balloon structure,
said balloon structure being of predominant longitudinal extent with a proximal end and a distal end, and being suitable for performing an expansion in an object to be dilated,
the balloon structure comprising a wall which has, transverse the longitudinal extent, at any point, an annular cross-section delimited externally by an outer surface which, at least in an intermediate portion thereof, is suitable for coming into contact with the object to be dilated, and internally by an inner surface which delimits an inflation chamber, in which:
at least one wall cavity is provided in the wall and is formed within the annular cross-section which delimits the inflation chamber so as to be disposed between the outer surface and the inner surface
the cavity extending, without interruptions and/or openings, longitudinally relative to the balloon structure between the proximal end and the distal end so that, when the balloon structure is inflated or expanded, the outer surface of the intermediate portion has, in cross-section transverse the longitudinal extent of the balloon structure, uniform curvature around the entire annular extent of the cross-section.

106. A catheter according to claim 105, which includes an apex tip which has a proximal end and an apex end and comprises:
a tubular apex portion disposed in the vicinity of the apex end, and
a proximal connecting tube disposed in the vicinity of the proximal end, in which:
the proximal connecting tube is partially housed with a distal portion thereof inside a proximal portion of the apex tube,
the connecting tube being connected to the apex tube so as to form a cavity which extends without interruption from an opening disposed at the proximal end of the connecting tube to an opening disposed at the apex end of the apex tube,
the connecting tube being suitable for connection to a wall cavity suitable for housing a guide wire, the wall cavity being provided within a wall which delimits a balloon inflation chamber.

107. A catheter according to claim 105, comprising an apex tip which has a proximal end and an apex end and comprises:
a tubular apex portion disposed in the vicinity of the apex end,
a proximal connecting tube disposed in the vicinity of the proximal end, and
a tube for anchoring a thrust wire or rod, also disposed in the vicinity of the proximal end, in which:
the anchoring tube and the proximal connecting tube are arranged at least partially side by side and are housed with respective distal portions inside a proximal portion of the apex tube, the connecting tube being connected to the apex tube so as to form a cavity which extends without interruption from an opening disposed at the proximal end of the connecting tube to an opening disposed at the apex end of the apex tube,
the connecting tube being suitable for connection to a wall cavity suitable for housing a guide wire, the wall cavity being provided within a wall which delimits a balloon inflation chamber, the anchoring tube being suitable for connection to a distal opening of a balloon structure for the leaktight closure thereof and for the anchorage of a distal end of a thrust wire provided inside the balloon.

108. A catheter according to claim 107 in which the anchoring tube, the connecting tube, and the apex tube are welded to form a single body.

109. A catheter according to claim 105 including an apex tip which has a proximal end and an apex end and comprises:
a tubular apex portion disposed in the vicinity of the apex end,
a first proximal connecting tube disposed in the vicinity of the proximal end, and
a second connecting tube, also disposed in the vicinity of the proximal end, in which:
the first connecting tube and the second connecting tube are arranged at least partially side by side and are housed with respective distal portions inside a proximal portion of the apex tube, the first and second connecting tubes being connected to the apex tube so as to form cavities which extend without interruption from respective openings disposed at the proximal ends of the connecting tubes to at least one opening disposed at the apex end of the apex tube, the first connecting tube being suitable for connection to a wall cavity suitable for housing a guide wire, the wall cavity being provided within a wall which delimits a balloon inflation chamber,
the second connecting tube being suitable for connection to a guide-wire duct suitable for housing a guide wire, the guide-wire duct being disposed within the balloon structure.

110. A catheter according to claim 109, in which the first and second connecting tubes and the apex tube are welded to form a single body.

111. A catheter according to claim 95, in which the thrust wire is disposed inside the catheter shaft connected proximally to the balloon.

112. A catheter according to claim 105, in which the guide-wire duct is disposed inside the catheter shaft which is connected proximally to the balloon.

113. A catheter according to claim 105, in which the wall portion which separates the wall cavity from the outer surface has an opening which forms a lateral aperture for allowing a guide wire to be inserted in the wall cavity or to emerge therefrom.

114. A catheter according to claim 105, in which the balloon structure is connected proximally to a shaft comprising an inflation cavity connected to the inflation chamber in a leaktight manner for the flow of a fluid from the shaft to the inflation chamber and vice versa.

115. A catheter according to claim 105, in which the balloon structure is connected proximally to a shaft comprising a guide-wire cavity connected to the wall cavity in a leaktight manner for the passage of a guide wire.

116. A catheter according to claim 115, in which the guide-wire cavity is disposed in the wall of the shaft and is separated from the outer surface of the shaft by a wall portion.

117. A catheter according to claim 116, in which the shaft has an opening in the wall portion which separates the guide-wire cavity from the outer surface, the opening being suitable for the passage of a guide wire.

118. A catheter according to claim 105, in which the balloon structure is connected proximally to a shaft comprising a guide-wire cavity connected to the wall cavity in a leaktight manner for the passage of a guide wire, the guide-wire cavity being provided in a guide-wire duct provided inside the shaft.

119. A catheter according to claim 118, in which the duct is connected in a leaktight manner to a lateral opening provided in the outer wall of the shaft to allow a guide wire to be inserted in the guide-wire cavity of the guide-wire duct or to emerge therefrom.

120. A catheter according to claim 105, in which the shaft has a plurality of portions formed with a plurality of ducts.

121. A catheter according to claim 105, in which the shaft has a plurality of portions formed with ducts made of different materials and/or with different thicknesses.

122. A catheter according to claim 105, in which the shaft has an inflation cavity that is in flow communication with the inflation chamber of the balloon structure, and a thrust wire having a distal end and a proximal end.

123. A catheter according to claim 122, in which the thrust wire is inside the inflation cavity.

124. A catheter according to claim 122, in which the thrust wire extends along the entire length of the shaft.

125. A catheter according to claim 122, in which the thrust wire is anchored by its distal end to the balloon structure.

126. A catheter according to claim 122, in which the thrust wire is anchored by its distal end to the tip of the catheter.

127. A catheter according to claim 122, in which the thrust wire is connected by its proximal end to an inner tube that is present in the shaft.

128. A tip for a catheter such as a catheter for angioplasty or for depositing an endolumenal prosthesis or stent in a duct, for example, a vascular duct, the tip having a proximal end and an apex end and comprising:
a tubular apex portion disposed in the vicinity of the apex end, and
a proximal connecting tube disposed in the vicinity of the proximal end, in which:
the proximal connecting tube is partially housed with a distal portion thereof inside a proximal portion of the apex tube,
the connecting tube being connected to the apex tube so as to form a cavity which extends without interruption from an opening disposed at the proximal end of the connecting tube to an opening disposed at the apex end of the apex tube,
the connecting tube being suitable for connection to a wall cavity suitable for housing a guide wire, the cavity being provided within a wall which delimits a balloon inflation chamber,
the apex tube being connected proximally in a leaktight manner to a distal opening of a balloon structure for the leaktight closure thereof.

129. A tip for a catheter such as a catheter for angioplasty or for depositing an endolumenal prosthesis or stent in a duct, for example, a vascular duct, the tip having a proximal end and an apex end and comprising:
an apex tubular portion disposed in the vicinity of the apex end,
a proximal connecting tube disposed in the vicinity of the proximal end, and
a tube for anchoring a thrust wire or rod, also disposed in the vicinity of the proximal end, in which:
the anchoring tube and the proximal connecting tube are arranged at least partially side by side and are housed with respective distal portions inside a proximal portion of the apex tube, the connecting tube being connected to the apex tube so as to form a cavity which extends without interruption from an opening disposed at the proximal end of the connecting tube to an opening disposed at the apex end of the apex tube,
the connecting tube being suitable for connection to a wall cavity suitable for housing a guide wire, the cavity being provided within a wall which delimits a balloon inflation chamber,
the anchoring tube being closed distally in a leaktight manner and suitable for connection to a distal opening of a balloon structure for the leaktight closure thereof and for the anchorage of a distal end of a thrust wire.

130. A tip according to claim 129 in which the anchoring tube, the connecting tube, and the apex tube are welded to form a single body.

131. A tip for a catheter such as a catheter for angioplasty or for depositing an endolumenal prosthesis or stent in a duct, for example, a vascular duct, the tip having a proximal end and an apex end and comprising:
a tubular apex portion disposed in the vicinity of the apex end,
a first proximal connecting tube disposed in the vicinity of the proximal end, and
a second connecting tube, also disposed in the vicinity of the proximal end, in which:
the first connecting tube and the second connecting tube are arranged at least partially side by side and are housed with respective distal portions inside a proximal portion of the apex tube,
the first and second connecting tubes being connected to the apex tube so as to form cavities which extend without interruption from respective openings disposed at the proximal ends of the connecting tubes to at least one opening disposed at the apex end of the apex tube,
the first connecting tube being suitable for connection to a wall cavity suitable for housing a guide wire, the cavity being provided within a wall which delimits a balloon inflation chamber,
the second connecting tube being suitable for connection to a guide-wire duct suitable for housing a guide wire, the guide-wire duct being disposed inside the balloon structure.

132. A tip according to claim 131, in which the first and second connecting tubes and the apex tube are welded to form a single body.

133. A method for the production of a tip,
said tip having a proximal end and an apex end and comprising:
a tubular apex portion disposed in the vicinity of the apex end, and
a proximal connecting tube disposed in the vicinity of the proximal end, in which:
the proximal connecting tube is partially housed with a distal portion thereof inside a proximal portion of the apex tube,
the connecting tube being connected to the apex tube so as to form a cavity which extends without interruption from an opening disposed at the proximal end of the connecting tube to
an opening disposed at the apex end of the apex tube,
the connecting tube being suitable for connection to a wall cavity suitable for housing a guide wire, the cavity being provided within a wall which delimits a balloon inflation chamber,
the apex tube being connected proximally in a leaktight manner to a distal opening of a balloon structure for the leaktight closure thereof;
said method provides for the steps of:
providing an apex tube having a proximal end and a distal end in which an apex opening is provided,
widening a proximal portion of the apex tube,
providing a connecting tube suitable for insertion in a wall cavity of a balloon structure, the wall cavity being suitable for housing a guide wire,
inserting a distal portion of the connecting tube in the widened portion of the apex tube so as to form a continuous cavity between a proximal opening of the connecting tube and the apex opening of the apex tube.

134. A method according to claim 133, which provides for the further step of:
providing an anchoring tube to be inserted with a distal portion thereof in the apex tube so as to be disposed at least partially beside the connecting tube, the anchoring tube being suitable for anchoring a thrust wire and being suitable for the leaktight closure of a distal opening of an inflation chamber of a balloon structure.

135. A method according to claim 133, which provides for the further step of:
providing a second connecting tube to be inserted with a distal portion thereof in the apex tube so as to be disposed at least partially beside the first connecting tube, the connecting tube being suitable for forming a second guide-wire cavity between a proximal opening of the second connecting tube and the apex opening of the apex tube, and being suitable for the leaktight closure of a distal opening of an inflation chamber of a balloon structure.

136. A balloon catheter, such as a catheter for angioplasty or for depositing an endolumenal prosthesis or stent in a duct, for example, a vascular duct, comprising:
a shaft in which an inflation cavity is provided, the inflation cavity having a proximal end and a distal end,
an inflatable balloon having a proximal end and a distal end, delimiting an inflation chamber, in which the distal end of the inflation cavity opens in flow communication with the inflation chamber,
a tip which closes the distal end of the balloon in a leaktight manner and has a connecting duct provided with a cavity which has a proximal end and an apex end,
the balloon comprising a wall which has, transverse the longitudinal extent, at any point, an annular cross-section delimited externally by an outer surface which, at least in the intermediate portion, is suitable for coming into contact with an object to be dilated, and internally by an inner surface which delimits an inflation chamber,
in which:
at least one cavity is provided in the wall, and is formed, for its entire extent in the wall of the balloon, within the annular cross-section which delimits the inflation chamber so as to be disposed between the outer surface and the inner surface, and
the cavity extends without interruptions and/or openings, longitudinally relative to the balloon structure, between the proximal end and the distal end of the balloon.

137. A catheter according to claim 136, in which a thrust wire is provided in the inflation cavity.

138. A catheter according to claim 137, in which the thrust wire extends with its distal end as far as the tip of the catheter.

139. A catheter according to claim 138, in which the distal end of the thrust wire is anchored to the tip.

140. A catheter according to claim 136, in which, when the balloon structure is inflated or expanded, the outer surface of the intermediate portion has, in cross-section transverse the longitudinal extent of the balloon structure, uniform curvature around the entire annular extent of the cross-section.

141. A method for the use of a catheter comprising a balloon structure
said balloon structure being of predominant longitudinal extent with a proximal end and a distal end, and being suitable for performing an expansion in an object to be dilated,
the balloon structure comprising a wall which has, transverse the longitudinal extent, at any point, an annular cross-section delimited externally by an outer surface which, at least in an intermediate portion thereof, is suitable for coming into contact with the object to be dilated, and internally by an inner surface which delimits an inflation chamber, in which:
at least one wall cavity is provided in the wall and is formed within the annular cross-section which delimits the inflation chamber so as to be disposed between the outer surface and the inner surface
the cavity extending, without interruptions and/or openings, longitudinally relative to the balloon structure between the proximal end and the distal end so that, when the balloon structure is inflated or expanded, the outer surface of the intermediate portion has, in cross-section transverse the longitudinal extent of the balloon structure, uniform curvature around the entire annular extent of the cross-section;
said method provides for the steps of:
inserting a guide wire in a vessel which is to be operated on,
fitting a catheter, provided with the balloon structure, on the guide wire, passing the wire through an apex guide-wire aperture, sliding it through a guide-wire wall cavity which, at least for its section corresponding to the extent of the balloon, is disposed in the balloon wall, and causing the wire to emerge from a proximal aperture, relative to the balloon,
inserting the catheter in the vessel, passing it along the guide wire until the balloon is disposed in the operation zone.

142. A method according to claim 141, which provides for the further step of:
advancing the catheter on the guide wire by pushing it by means of a thrust wire provided in the catheter body.
